(19) United States
(12) Patent Application Publication
(10) Pub. No.: US 2010/0307511 A1
(43) Pub. Date: Dec. 9, 2010

(54) Mandibular Advancement Appliance

(76) Inventor: Thomas E. Meade, Albuquerque, NM (US)

Correspondence Address: BROWDY AND NEIMARK, P.L.L.C.;624 NINTH STREET, NW, SUITE 300, WASHINGTON, DC, 20001-5303, US

(21) Appl. No.: 12/663,177

(22) PCT Filed: Apr. 23, 2007
(86) PCT No.: PCT/US07/67195

§ 371 Date: Aug. 24, 2010

(51) Int. Cl. A61F 5/56

(52) U.S. Cl. 128/848

(57) ABSTRACT

A mandibular advancement device for pulling the jaw forward includes upper and lower plastic trays conforming to upper teeth and maxillary dentition soft tissue and palate and lower teeth and mandibular dentition and soft tissue, respectively. A ball type of hook support is located on both sides of the upper tray at a forward position and a plurality of ball type of hook supports are located at a rearward position of both sides of the lower tray. Tension coil springs are fitted to each of the upper tray ball hook supports and to one of the plurality of lower ball type of hook supports. The plurality of lower tray ball hook supports provides discrete levels of tension force of the coil spring on each side of the appliance. A plastic tube may be fitted over the coil springs to prevent injury to the inside of a user's mouth.

### FIELD

This invention is related to oral appliances for preventing or alleviating snoring and sleep apnea. The appliance is a removable mandibular advancement device that uses coil springs to pull the lower jaw forward during use.

### BACKGROUND

It is known in the art that an oral appliance that serves to move the mandible forward reduces sleep apnea and snoring of a user. A variety of appliances are available that are removable and advance the mandible. Some of these appliances are adjustable to provide different mandible advancement forces.

One such appliance issued to Frantz et al on Aug. 29, 2000, U.S. Pat. No. 6,109,265, uses elastic bands hooked between upper and lower trays to pull the mandible forward. The elastic urethane bands are interchangeable with other bands having varying lengths and/or elasticity to provide different advancement forces on the mandible. This appliance uses rubber bands, which tend to break with use, and requires new rubber bands to be applied to change the tension.

Other such appliances are disclosed in U.S. Pat. Nos. 4,505,672 to Kurz, 5,775,219 to Thronton, 6,450,167 to David et al., 5,947,724 to Frantz et al., 6,729,335 to Halstrom, and 5,467,783, 5,682,903, and 6,055,986 to Meade.

### SUMMARY

The present invention is a removable, adjustable oral appliance for alleviating snoring and sleep apnea in a user. The appliance has tension springs fitted between ball type hook supports on both sides of an upper teeth conforming tray and extending in tension to one of a plurality of ball type hook supports on the teeth conforming lower tray. These springs provide discrete levels of tension force to the lower jaw to advance the jaw forward.

### BRIEF DESCRIPTION OF THE DRAWINGS

FIG. 1 is a side view of the upper and lower trays of the invention with the tension coil springs in place.

FIG. 2 is a side view of a coil spring having a protective plastic sleeve thereon used in the invention, and

FIG. 3 is an exploded view of the invention.

### DETAILED DESCRIPTION

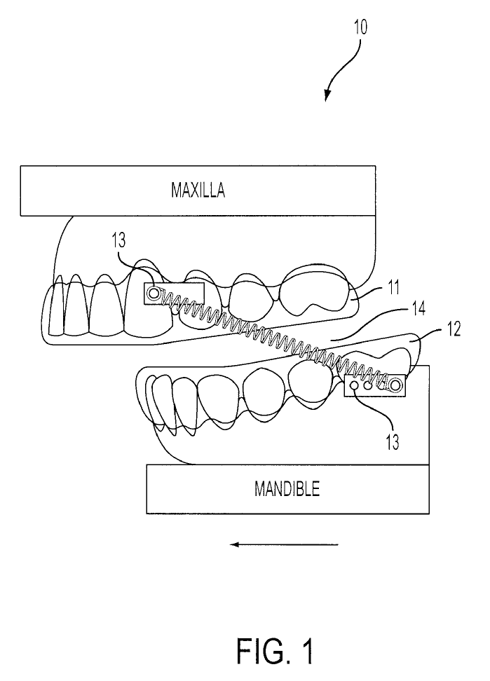

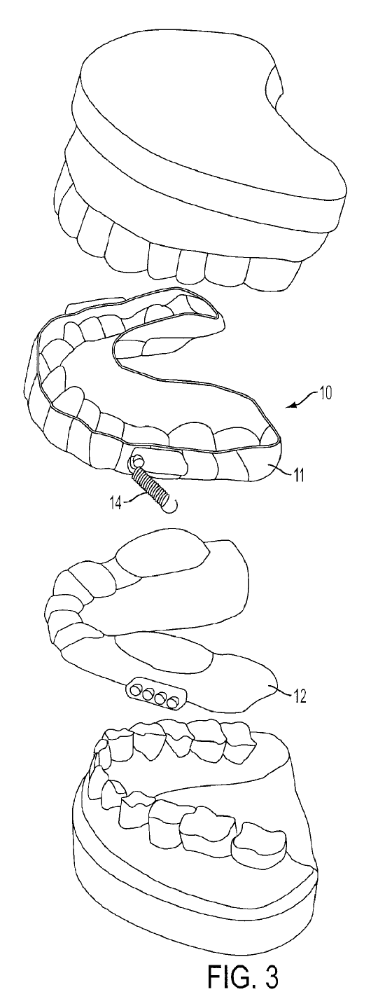

Referring to FIGS. 1 and 3 the appliance 10 includes an upper tray 11 that conforms with the user's upper teeth including maxillary dentition soft tissue and palate and includes a ball type hook support 13 on each side of the tray, and a lower tray 12 that conforms to the user's lower teeth including mandibular dentition and soft tissue and that includes a plurality of ball type hook supports 13 on each side of the tray. In one embodiment, there are four ball type hook supports on each side of the lower tray 12. The upper hook support on each side of the upper tray is attached at a forward portion of the tray, and the plurality of hook supports on each side of the lower tray are attached at a rearward portion of the tray. The upper and lower trays are injection molded and made of acrylic plastic. Other materials may be used, as well, which have similar properties to the acrylic plastic. The upper and lower trays may also be laboratory fabricated with the spherical ball attached. The balls may be in injection molded and attached to the upper and lower trays with an adhesive.

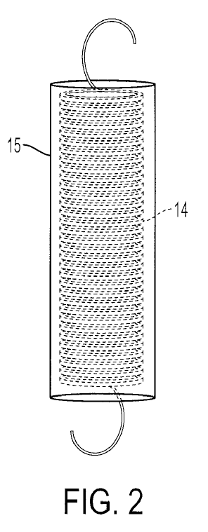

Each of the hook supports 13 have smooth spherical balls and a stem extending therefrom that fits into corresponding holes in the upper and lower trays and are bonded thereto. Each spring 14 shown in FIG. 2 may be made of plastic or stainless steel and has a curved hook at each end. A sleeve 15 as seen in FIG. 2 may be positioned over each spring to guard against injury to a user's gums. The sleeve 15 may be made of plastic or other suitable material.

In use, a doctor would determine the proper strength coil springs to be used and would hook one end of the coil springs to the hook support on each side of the upper tray and hook the other end of the coil springs to one of the plurality of hook supports on each side of the lower tray. The particular hook support used to hook the spring ends on the lower tray would be determined by the doctor to provide a predetermined tension force to each side of the lower tray to advance the mandible forward under the proper tension in the direction of the arrow seen in FIG. 1.

The appliance of the invention provides many advantages over prior art appliances including posterior support for all posterior teeth, complete vertical and lateral freedom of movement of the mandible, no sharp edges that may injure a user, and easy adjustment of the mandible advancement force. Furthermore, the tension springs are less likely to break as compared with elastic bands.

It is to be understood that the phraseology or terminology employed herein is for the purpose of description and not of limitation. The means, materials, and steps for carrying out various disclosed functions may take a variety of alternative forms without departing from the invention.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation

What is claimed is:

1. An oral appliance for use in advancing a user's lower jaw, comprising:
upper and lower trays that fit to a user's upper and lower teeth,
a pair of hook supports attached one on each side of the forward portion of the upper tray,
a plurality of ball type hook supports attached to each side of the rear portion of the lower tray, and
a pair of tension coil springs, each spring hooked at one end thereof to the hook support at each side of the upper tray, and hooked to one of the plurality of hook supports at each side of the lower tray,
whereby the spring force of each coil spring on the lower tray may be adjusted by moving the hooked end on each side of the lower tray to another hook support.

2. The oral appliance of claim 1, wherein each hook support comprises a spherical member having a stem extending therefrom, the stem fitting into a corresponding hole in the upper and lower trays.

3. The oral appliance of claim 1, further comprising a plastic sleeve positioned over each of the coil springs.

4. The oral appliance of claim 1, wherein the plurality of ball type hook supports comprises four hook supports on each side of the lower tray.

5. The oral appliance of claim 1, wherein coil springs are made of stainless steel.

6. The oral appliance of claim 1, wherein the upper and lower trays are injection molded acrylic members.
